(19) United States
(12) Patent Application Publication
(10) Pub. No.: US 2024/0345233 A1
(43) Pub. Date: Oct. 17, 2024

(54) AUTONOMOUS IMAGING SYSTEM ON CHIP

(71) Applicant: Exo Imaging, Inc., Santa Clara, CA (US)

(72) Inventors: Kutay ÜSTÜNER, Mountain View, CA (US); Anming CAI, Santa Clara, CA (US); Chad STEWARD, Santa Clara, CA (US)

(21) Appl. No.: 18/634,712

(22) Filed: Apr. 12, 2024

Related U.S. Application Data
(60) Provisional application No. 63/459,905, filed on Apr. 17, 2023.

(51) Int. Cl. G01S 7/52; G01S 15/89

(52) U.S. Cl. CPC G01S 7/52022; G01S 7/52087; G01S 15/89

(57) ABSTRACT

An ultrasound imaging system integrated on a chip for autonomous scanning includes an on-chip input memory configured to store scan sequence instructions and parameters, an on-chip processor configured to read the scan sequence instructions and parameters in the input memory, and an on-chip beamformer configured to be programmed and timed by the processor according to the scan sequence instructions and parameters. In some examples, the scan sequence instructions and parameters are stored in a nonvolatile input memory programmed at the factory. In other examples, they are received from an external user device. The scan parameters are optimized or minimized to accommodate the limited input memory capacity. In some examples, the scan sequence instructions and parameters include programmable nested loops, and each of the programmable nested loops corresponds to a transmit and/or receive event in a scan sequence.

### CROSS-REFERENCE TO RELATED APPLICATIONS

The present application claims priority to U.S. Provisional Patent Application No. 63/459,905, titled “AUTONOMOUS IMAGING SYSTEM ON CHIP” filed Apr. 17, 2023, which is related to U.S. patent application Ser. No. 17/569,805, titled “FULL-ARRAY DIGITAL 3D ULTRASOUND IMAGING SYSTEM INTEGRATED WITH A MATRIX ARRAY TRANSDUCER” filed Jan. 6, 2022. Each application is incorporated by reference herein in its entirety.

### TECHNICAL FIELD

The present inventions relate to systems, devices, and methods for ultrasound imaging, more specifically, for an Imaging System on Chip (iSoC) that performs scanning and imaging autonomously without the need for real-time control by an external processor.

### BACKGROUND

Ultrasound imaging is an imaging method that uses sound waves to produce images of structures within a patient's body. Because ultrasound images are captured in real-time, they can also show movement of the body's internal organs as well as blood flowing through the blood vessels. The images can provide valuable information for diagnosing and directing treatment for a variety of diseases and conditions.

Wide field of view 3D imaging with large steering angles generally requires two-dimensional (2D) (matrix) array transducers with high element density both in azimuth and elevation. High resolution and high sensitivity, on the other hand, generally require wide apertures. Therefore, a good 3D transducer generally requires a very high transducer element count on the order of thousands to tens of thousands of (transducer) elements. High element count creates a major implementation challenge for imaging systems, particularly for receiving beam formation, forcing to keep the element count low, and/or limiting the receive beam formation to a multi-step beam formation where only the first step, the microbeamformer, is in close proximity to the array or is integrated with it and the second step, the macrobeamformer, is on a remote processor. The microbeamformer generally performs the intra-subarray beamformation and is typically a single-beam analog beamformer often without dynamic focusing capability. The macrobeamformer performs the inter-subarray beamformation and is typically a digital beamformer with dynamic focusing and multibeam (parallel beam) capabilities. The split processing can create issues of connectivity via flex/cables and limits the signal and control data bandwidth.

General-purpose processors and Field Programmable Gate Arrays (FPGAs) consume a lot more power than a wearable product can afford.

### SUMMARY

To operate autonomously, an Imaging System on Chip needs a low power but fully capable central controller that is integrated into the application specific integrated circuit (ASIC). This in turn requires the central controller to have a streamlined ultrasound-specific architecture and a streamlined set of input parameters to reduce on-chip storage needs.

In some approaches, a matrix array transducer is integrated with a transmit and receive beamformer packed in an application specific integrated circuit (Imaging System on Chip, iSoC) that has a digital acquisition channel per transducer element. This helps reduce the cost, size, weight, and power of an ultrasound imaging system, add functionality (e.g., real-time 3D) and improve performance. An on-ASIC delay and weight (apodization) computer for on-chip generation of delay and apodization for transmit and receive beamformation using a few parameters per beam is also provided. This eliminates the need for precomputing and storing the delay and apodization profiles which may require a big memory particularly for a matrix array transducer used for 3D imaging.

To support wide range of clinical applications, ultrasound imaging systems are capable of performing imaging in a variety of modes (e.g., B-Mode, Color Doppler, Spectral Doppler, M-Mode, Elastography, etc.) with a variety of features (e.g., Pulse Inversion, Frequency Compounding, Spatial Compounding, etc.) in a variety of scan geometries (sector, vector, trapezoid, linear, steered linear, cone, rectangular prism, etc.), in a variety of number of dimensions (1D, 2D, 3D, 4D). To support this rich set of functionality, imaging systems employ a software-controlled general-purpose processor (central processing unit (CPU), graphics processing unit (GPU)) or a Field Programmable Gate Array (FPGA) that runs complex mode- and feature-specific scan sequence algorithms and state machines for the acquisition of data and updates the parameters of the transmit and receive beamformer and other signal processing blocks per pulse-echo event. The integration of the on-chip central controller for imaging devices has been a challenging task. In addition, the complexity introduced by the large number of parameters required for each imaging mode was left unsolved for the on-chip controller.

The present disclosure describes a method and system for an Imaging System on Chip (iSoC) capable of scanning and imaging autonomously without the need for real-time control by an external processor. This enables the development of low cost, low power, small and lightweight imaging products that can be worn by patients for diagnosis, monitoring or therapy.

In some examples, methods and systems to enable an Imaging System on Chip to scan and image autonomously in any mode with any feature, in any scan geometry and in any number of dimensions, without real-time control by an external processor are disclosed herein.

In a preferred embodiment, the Imaging System on Chip has an on-ASIC input memory that stores the instruction set for the scan sequence, and the timing and imaging parameters for each event in the scan sequence. The scan instructions and parameters that together define a scan sequence are called a Scan Design. In this preferred embodiment, the Imaging System on Chip also has an ultrasound-specific central controller that can generate a scan sequence based on the instructions in the input memory and execute each transmit and receive event in the scan sequence with the desired timing and imaging parameters also captured in the input memory. In a preferred embodiment the Scan Designs are made of programmable nested loops in space and time dimensions of transmit and receive events where each event or event loop is preceded by imaging and timing parameters to be updated at that point in the scan sequence. In a preferred embodiment the imaging parameters are streamlined to minimize on-ASIC storage requirements for Scan Designs.

In some aspects, an ultrasound imaging system integrated on a chip for autonomous scanning includes an on-chip processor configured to read scan sequence instructions and parameters. In some examples, the ultrasound imaging system also includes an on-chip input memory to store the scan sequence instructions and parameters. In some examples, the ultrasound imaging system includes an on-chip beamformer configured to be programmed and timed by the processor according to the scan sequence instructions and parameters. In some examples, the scan sequence instructions and parameters are received from an external user device. The scan parameters are optimized or minimized to accommodate the limited input memory capacity. In some examples, the scan sequence instructions and parameters include programmable nested loops, and each of the programmable nested loops corresponds to a transmit and/or receive event in a scan sequence.

The scanning autonomously executed by an on-chip center controller includes a periodically repeated sequence of events. In some examples, the scan sequence may include individual events, temporal loops of events, spatial loops of events in x and y, where each event and the loop of events from the inner most event loop, x loop, and y loop, to the outermost scan loop are timed.

For example, when a user sends the instruction, e.g., from an external processor to the on-chip central controller to start a scanning process, the on-chip central controller autonomously executes the scan sequence instructions and parameters, collectively called scan design, already stored in the internal input memory without any further intervention from the user. The user can pause, resume or stop the scanning. A scan design includes scan sequence instructions and the parameters for each transmit and/or receive event, and the loops of events that comprise the scan sequence. The scan design also defines the timing, inter event, and/or inter loop periods of a full frame/plane for 2D imaging, two planes for bi-plane imaging, and full volume for 3D imaging. A scan design may function as a framework for the autonomous scanning process. The central controller drives the execution of the scan design. Multiple scan designs each customized for a different imaging mode, feature, or clinical application may be stored in the input memory and selected by the user per the need.

In contrast to scanning process in some other approaches, e.g., software running on power consuming CPUs, GPUs, FPGAs or a combination which utilizes mode specific and scan geometry specific program, the subject technology disclosed herein provides a mode/feature and scan geometry agnostic scan design/framework to flexibly fit all possible scanning requirements into an ASIC-executed autonomous scanning.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

The systems, methods and devices of this disclosure each have several innovative aspects, no single one of which is solely responsible for the desirable attributes disclosed herein.

It is to be understood that both the foregoing general description and the following detailed description are exemplary and explanatory and are intended to provide further explanation of the subject technology.

Note that the various embodiments described above can be combined with any other embodiments described herein. The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes and may not have been selected to delineate or circumscribe the inventive subject matter.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates an exemplary architecture of the components of an Autonomous Imaging System on Chip (AiSoC), according to some embodiments.

FIG. 2 shows an exemplary schematic diagram of an ultrasonic system using a transducer assembly comprised of a 2D array of transducers and an ASIC mounted on a PCB with additional circuitry, and a remote processor with a user interface and display, according to some embodiments.

FIG. 3 shows a schematic of a digital 3D single-stage full-array beamformer with an ASIC, according to some embodiments.

FIG. 4 shows a schematic of a digital 3D two-stage full-array beamformer with an ASIC, according to some embodiments.

FIG. 5 shows a graph of the geometry of ultrasound beams generated by an ultrasound transducer array, according to some embodiments.

FIG. 6 shows a flow chart of a 3D dynamic delay and weight computer, according to some embodiments.

FIG. 7 shows a graph of the geometry of ultrasound beams generated by an ultrasound transducer array, according to some embodiments.

FIG. 8A and FIG. 8B illustrate an exemplary rectangular beam grid for sampling a truncated pyramid, according to some embodiments.

FIG. 9 illustrates an exemplary scan design, according to some embodiments.

Reference will now be made to implementations, examples of which are illustrated in the accompanying drawings. In the following description, numerous specific details are set forth in order to provide a thorough understanding of the present invention. However, it will be apparent to one of ordinary skills in the art that the present invention may be practiced without requiring some of these specific details.

### DETAILED DESCRIPTION

It is understood that various configurations of the subject technology will become readily apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, drawings and detailed description are to be regarded as illustrative in nature and not as restrictive.

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be apparent to those skilled in the art that the subject technology may be practiced without these specific details. In some instances, well-known structures and components are shown in block diagram form in order to avoid obscuring the concepts of the subject technology. Like components are labeled with identical element numbers for ease of understanding.

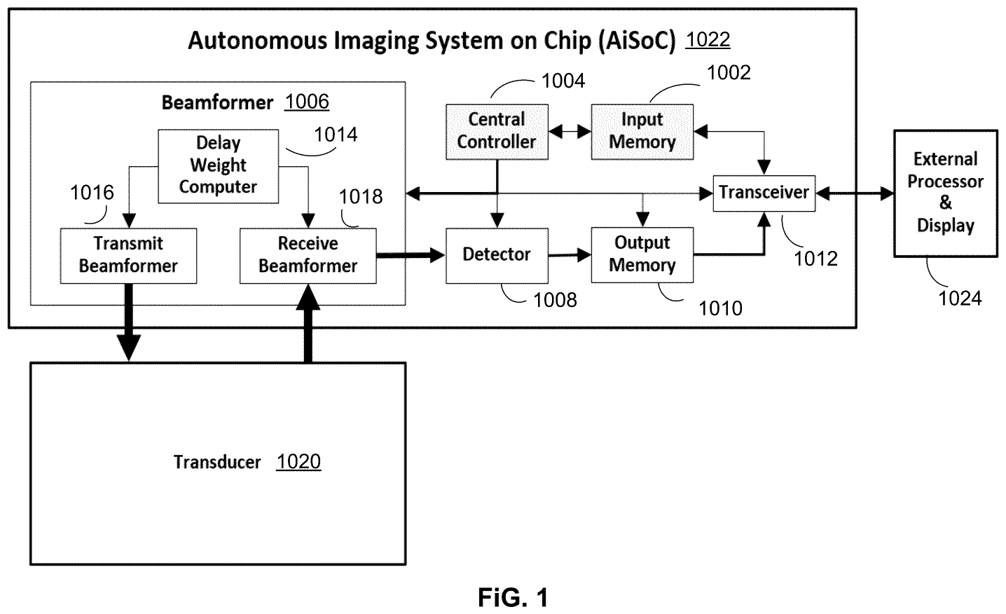

FIG. 1 illustrates an exemplary architecture of the components of an Autonomous Imaging System on Chip (AiSoC) 1022, according to some embodiments. The present disclosure is focused on the central controller and the input memory shown in FIG. 1.

Referring to FIG. 1, an Input Memory 1002 may store multiple Scan Designs selectable by the user. This memory 1002 can be a read only memory (ROM, or EEPROM) which would be programmable only at the factory by pre-designed scan sequences. Alternatively, it can be a random-access memory (RAM, SRAM, DDR SRAM) for storage of field upgradable scan sequences.

Autonomous scanning is performed by the Central Controller 1004 of the ASIC. It reads a Scan Design from the Input Memory 1002, executes the scan sequence instructions while dispatching the imaging parameters to the respective components in a synchronized manner based on the timing parameters also in the Scan Design. The Central Controller 1004 is agnostic to the scan geometry, mode, and feature. It executes Scan Designs faithfully without any knowledge of the use case.

The components of the AiSoC that the Central Controller 1004 controls may include a beamformer 1006, a detector 1008, an output memory 1010, and a transceiver 1012. The beamformer 1006 may have an on-chip Delay and weight (apodization) computer 1014, a transmit beamformer 1016 and a receive beamformer 1018. The transmit beamformer 1016 and a receive beamformer 1018 drive and receive from an array of transducer elements 1020. The AiSoC 1022 is preferably integrated with the transducer array for efficient and low-cost coupling to the transducer elements 1020. The transducer 1020 can be a 1-D, 1.25-D, 1.5-D, or 2-D array. It can be a Micromachined Electromechanical Sensor (MEMS) such as Piezoelectric or Capacitive Micromachined Ultrasound Transducer (pMUT or cMUT).

The detector 1008 may include a complex demodulator down to baseband followed by a decimator, an envelope detector and log compressor for B-Mode detection, a clutter filter, autocorrelator and flow parameter detector for Color Doppler Mode. The output memory 1010 provides a buffer for processed outputs of the beamformer for transmission to an external processor 1024 for further processing (e.g., volume rendering) and display. The transceiver 1012 can be wired, e.g., USB, or wireless, e.g., Bluetooth or Wi-Fi.

### Ultrasound Imaging System

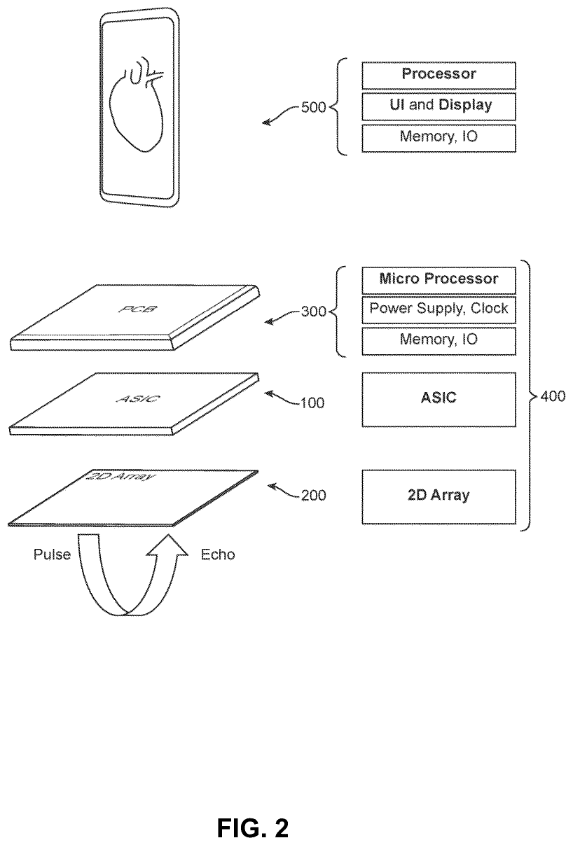

FIG. 2 shows an exemplary embodiment of the ultrasonic imaging system disclosed herein, according to some embodiments. The imaging system may include an application specific integrated circuit (ASIC) (100) preferably integrated with a transducer 200. The transducer may be a one-dimensional or a two-dimensional array of pMUT (piezoelectric micromachined ultrasound transducer), cMUT (capacitive micromachined ultrasound transducer), or bulk PZT elements. The ASIC and transducer array are typically mounted on a printed circuit board (PCB) (or PCBs) (300). The PCB may have additional circuitry such as an (optional) microprocessor, power supply (battery, regulators), clock, memory and an input output device.

The ASIC, transducer array, and the PCB form a transducer assembly (400). The area of the transducer assembly may match the area of the transducer array to keep the footprint small. The transducer assembly can be packaged in a patch, or in a wearable or holdable housing.

The transducer assembly, via an input output device, may communicate with a remote processor (500) that may include a user interface, display and memory. The processor may be a mobile device such as a smart phone, smart watch, pad or a laptop, or it can be a desktop computer. It may perform image processing, perform plane and volume rendering, and connect to a network and database such as electronic health records. The communication between the transducer assembly and the remote processor may be wired or wireless, using standard communication protocols.

In one example, the microprocessor on the transducer assembly may initialize the ASIC with a small set of parameters such as the imaging frequency and the transmit and receive f-numbers and then may provide the transmit and receive beam parameters (beam origin, angle, focus depth) for each pulse-echo (transmit-receive) event in the scan sequence. An on-ASIC delay and weight computer may compute the transmit and receive beamforming parameters (delay and weight) for each beam defined by the transmit and receive beam parameters. The ASIC may send out a steered and focused transmit pulse, may receive the echo from tissue at each transducer element, and may form receive beams using ASIC-computed delay and weight. The outputs of the ASIC are typically the fully formed beams using the full aperture.

In an alternative example, the microprocessor on the transducer assembly is optional and the microprocessor on the transducer assembly may help ASIC communicate with the remote processor to do field upgrades of the ASIC input memory, or to transfer the formed beams and gathered information to the remote processor for further processing, analysis or display. An on-ASIC central controller (e.g., 1004) executes a scan design defined by the scan sequence instruction set and imaging system parameters stored in the on-ASIC input memory forming frames or volumes of images periodically without real-time control from an external processor. The on-ASIC delay and weight computer may compute the static transmit and dynamic receive beamforming parameters (delay and weight) for each beam defined by the transmit and receive beam parameters in the scan design. The ASIC may send out a steered and focused transmit pulse, may receive the echo from tissue at each transducer element, and may form receive beams using ASIC-computed delay and weight. The outputs of the ASIC are typically the fully formed beams using the full aperture.

The sections below describe the transducer assembly, the transmitter and receiver, the geometry used for the derivation of a 3D delay equation, and a method and device for the delay and weight computation using the 3D delay equation.

### Transducer Assembly

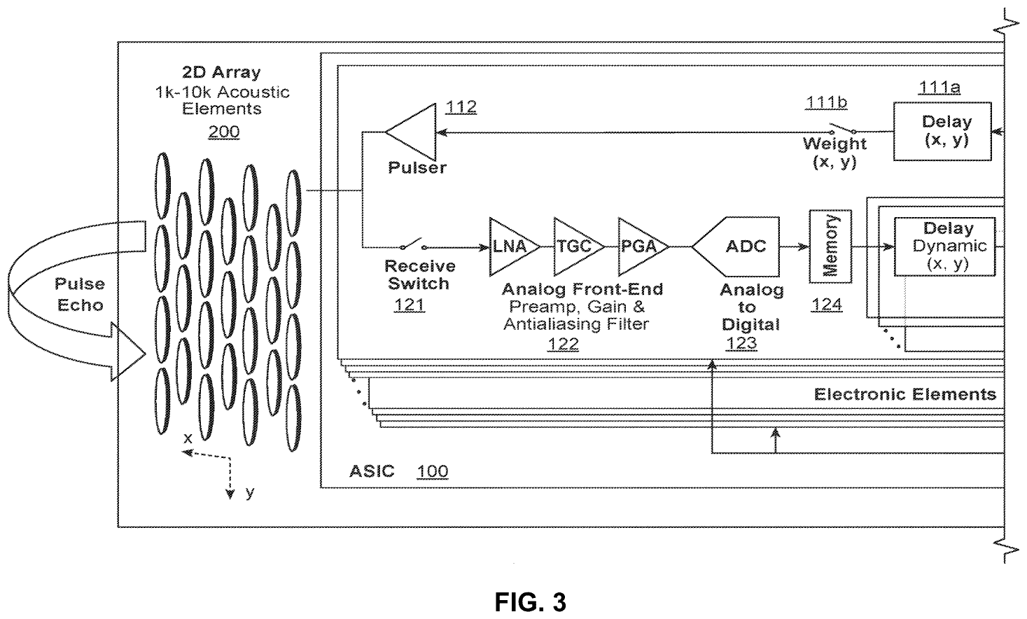

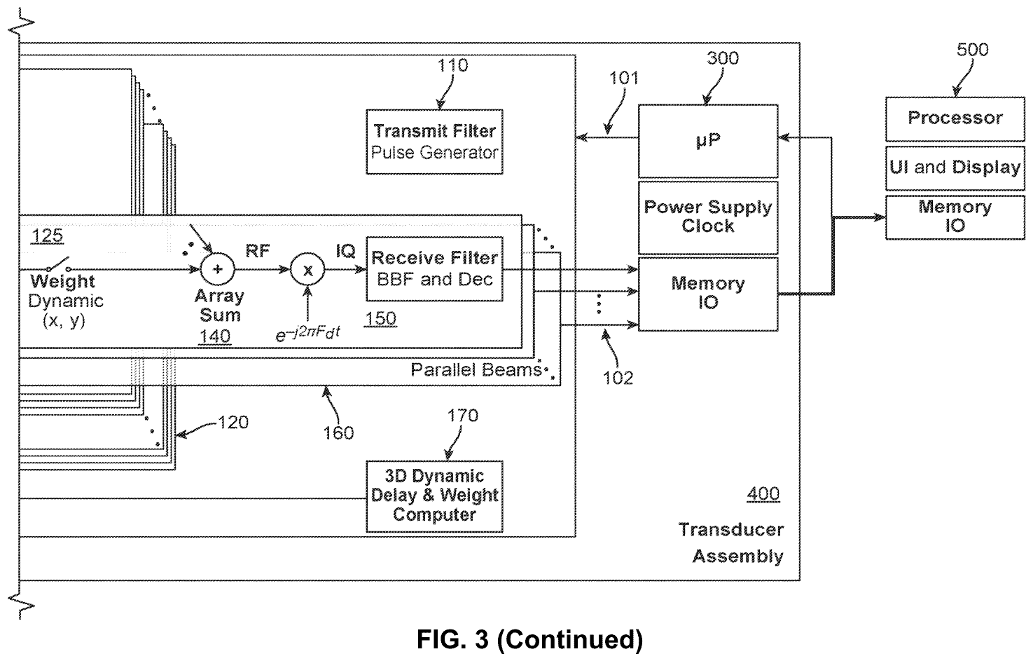

FIG. 3 shows the details of the transducer assembly (400), and the ASIC (100) within the transducer assembly, according to some embodiments.

In one example, the ASIC receives inputs (101) from the microprocessor on the PCB (300). The inputs may include initialization parameters such as transmit center frequency and bandwidth, transmit and receive f-number, and receive center frequency and bandwidth. The ASIC may also receive transmit and receive beam parameters and a trigger for each pulse-echo event. The transmitter may create the transmit pulse (110), apply an element coordinate dependent delay (111a) and weight (111b) to the pulse, and drive the pulser (112) of each acoustic element with the delayed and weighted pulse based on the transmit pulse and transmit beam parameters.

In an alternative example, the microprocessor on the PCB (300) is optional and the optional microprocessor on the PCB (300) may help ASIC communicate with the remote processor to do field upgrades of the ASIC input memory, or to transfer the formed beams and gathered information to the remote processor for further processing, analysis or display. An on-ASIC central controller (e.g., 1004) executes a scan design defined by the scan sequence instruction set and imaging system parameters stored in the on-ASIC input memory forming frames or volumes of images periodically without real-time control from an external processor. The on-ASIC delay and weight computer may compute the static transmit and dynamic receive beamforming parameters (delay and weight) for each beam defined by the transmit and receive beam parameters in the scan design. The ASIC may send out a steered and focused transmit pulse, may receive the echo from tissue at each transducer element, and may form receive beams using ASIC-computed delay and weight. The outputs of the ASIC are typically the fully formed beams using the full aperture.

The receive path for each acoustic element can contain a transmit/receive switch (121), an analog front end (122) for low noise preamplification, time gain compensation and antialiasing, an analog to digital converter (ADC) (123), an element memory (124), and a beamformer (125) that may apply time varying (dynamic) delay and weight on the stored element data. The transmit beamformer (delay and weight), pulser, receive switch, analog front-end, ADC, memory and receive beamformer (delay and weight) circuitry may form an electronic element (120). There may be an electronic element per acoustic element.

The outputs of the electronic elements can be summed across the whole array (140) to complete the full array beamformation. The beams thus formed may then be filtered by a receive filter (150) for data compression which may comprise demodulation to baseband by a complex time varying multiplier followed by a lowpass baseband filter (BBF). The delay, weight, array sum and receive filter circuitry may be duplicated to form multiple beams with distinct delay and/or weight parameters in parallel (160) using the same element data stored in the memory. The delay and weight for the transmit and receive beamformation (for all parallel beams) may be created by an on-ASIC 3D Dynamic Delay and Weight Computer (170). The outputs of the ASIC (102) may be the complex (in phase and quadrature phase) samples of parallel beams. The transducer assembly stores the output beams and sends them to a remote processor (500) for further processing, rendering, and display.

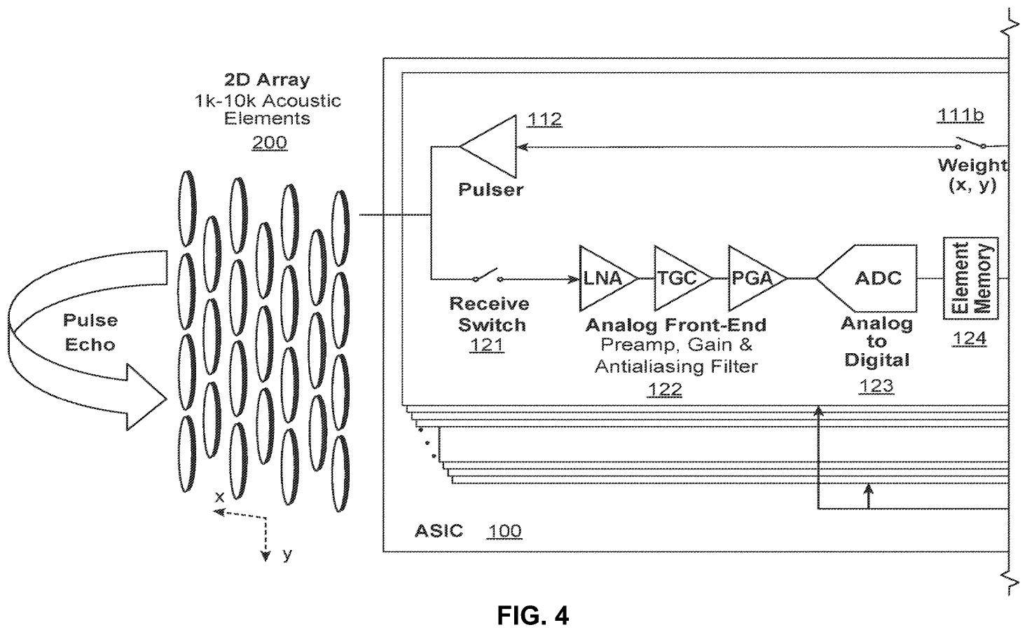

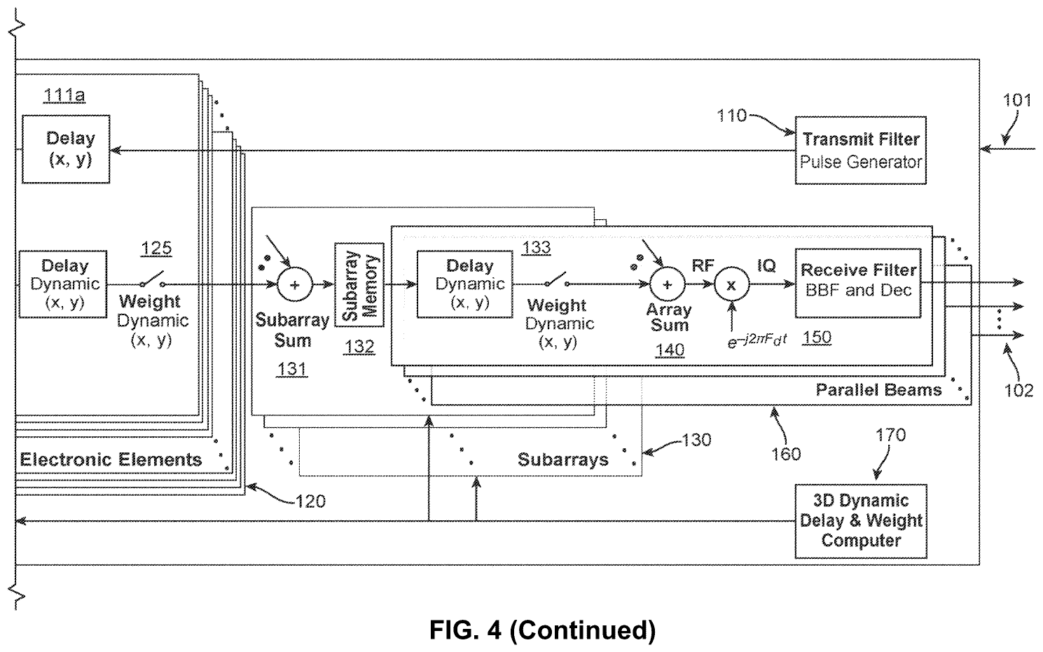

The receive beamformation of FIG. 3 can also be implemented in multiple stages. FIG. 4 shows a schematic of a digital 3D two-stage full-array beamformer with an ASIC, according to some embodiments. The multi-stage implementation allows flexibility to reduce the size of both the element memory and the parallel beam circuitry. Instead of summing the outputs of all electronic elements, the outputs of subsets of electronic elements (subarrays) (130) may be summed (131) and stored in a second set of subarray memory (132). The first stage beamformation within each subarray may also be known as micro beamformation. A second stage applies delay and weight (133) on the subarray beamformer outputs, and the array sum (140) may complete the full array beamformation. Only the second stage circuitry (macro beamformer) may be duplicated for parallel beam operation. The subarray size can be Sₓ×S_{y} elements where Sₓ and S_{y} can be 2, 3, 4, 5, etc., . . . electronic elements.

### Transmitter

A single K-bit deep, L-bit long shift register with a programmable clock can serve as an arbitrarily programmable pulse generator (110).

The depth of the shift register K may be determined by the number of pulser states. In general, a K-bit deep shift register can support pulsers up to 2^{K} states. So K would be 1 for 2-state (unipolar) pulsers, 2 for a 3-state (bipolar) and 4-state pulsers, and so on.

The length L of the shift register may be determined by the maximum pulse length spec and the transmitter clock frequency. In a preferred embodiment, the shift register length L is set to 256 bits. This would support up to 16-cycle long pulses at a transmit clock cycle that is 16 times the transmit center frequency. Longer than 16-cycle pulses can still be supported by lowering the transmitter clock frequency (trading off delay quantization step).

The simplest type of pulses may be unipolar pulses where the active node of the transducer element is altered between a ground and a positive (or negative) voltage rail by two complementary switches. These switches can be controlled by a single one-bit stream, a set of is for +V segment followed by a set of 0s for GND, where this pattern of is and 0s is repeated as many cycles as needed. Each bit may represent the duration of a transmitter clock cycle. So if transmitter clock cycle is 16 F₀ the bit stream of a two-cycle pulse at F₀ would be 11111111000000001111111100000000. The durations of the individual +V and GND segments can be fixed or independently programmable, e.g., for linear (or nonlinear) frequency modulation, or some other coded excitation. Such a bit pattern can be generated in advance and loaded to the pulse generator shift register in the ASIC during initialization and streamed out upon receiving the impulse indicating the start of transmission. In some embodiment, the start and/or end of the pulses can be marked by a very short code such as 010, e.g., 11111111000000001111111100000000010, to trigger other transmit and/or receive circuitry on or off. Utilization of such an embedded code may require a decoder (Matched Filter) of the same length. In some embodiments, the transmit/receive switch of each element may be turned on to the receive mode as soon as the element's own pulse transmission is complete without waiting for all elements to finish pulse transmission. This can help clean up some of the near field artifacts by temporally dispersing the leaked transmit and receive enable/disable signals and eliminate dead zones due to missed receive samples.

Next in complexity are the 3-state bipolar pulses where the active node of the transducer element is altered between a positive, ground, and a negative voltage rail by three complementary switches. This type of pulses can be implemented using a 2-bit deep pulse stream where for example 00 indicates ground, 10 indicates +V, and 01 indicates −V. The 11 state can be utilized to mark the start and/or end of the pulse.

A special case of the 3-state bipolar pulses is where the transducer is grounded only before the pulse starts and after the pulse ends and switched between the +V and −V states during the pulse. This type of pulse may offer the best second harmonic suppression compared to all 2-state pulses and those 3-state pulses with ground segments within the pulse. It may also be the simplest (lowest cost) architecture in terms of power supplies. This special case of bi-polar pulses can be implemented using a single bit stream above where 1 is mapped say to +V and 0 to the −V. The embedded snippet of code described above can be used to indicate the start of the ground state at the end of the pulse. Upon reception of this code the transducer element is grounded until the start of the next pulse which is indicated by a stream of 1's. The pulse inversion capability can be added with an additional programmable bit that is common to all elements that inverts the mapping of the 1 and 0 values to −V and +V at the pulser.

A pulse common to all elements can be generated upon an impulse marking the onset of a pulse-echo event which is typically repeated at regular Pulse Repetition Intervals (PRI). Then, the pulse may be delayed (11a) by an element specific delay for the elements (and in some embodiments, every element) of the array. The delayed pulse can then be weighed by an element specific weight for apodization. Here, a simple binary on/off weight is shown. In a preferred embodiment both the delay and weight of transmit beamformer are generated by the on-ASIC delay and weight computer (170) before the transmit event starts.

The output of the apodization can drive the transmit pulsers (112) after a digital to analog conversion.

In some embodiments, the pulse generator and delay operation share the same transmitter clock for architectural simplicity. Further, for efficiency purposes, the transmitter clock frequency F₃ can be varied as a function of the transmit center frequency F₀ and may be set equal to 16 F₀ to achieve a desired delay quantization step of T₀/16, where T₀=1/F₀.

In some embodiments, the order of the pulse generator, delay, and binary weight can be changed. For example, the binary weight can be moved before the delay operation or the delay operation can be moved before the pulse generator, etc., for various architectural tradeoffs.

### Receiver

A typical receiver applies a dynamically varying gain, delay, and weight (apodization) on the echo from individual elements sᵢⱼ(t), where (i, j) are the column and row indices of the elements of a matrix array. Then, the beamformer may sum the amplified, delayed, and weighted element signals to generate a beam b(r, θ, xₒ) where xₒ is the (xₒ,yₒ,zₒ) coordinates of the beam origin (zₒ is zero for planar arrays), r is depth and θ is the beam angle in z-x and z-y planes. For a digital beamformer, the analog signal may be converted to digital by an ADC after the LPF before the delay stage.

$$b ( r , θ , {x}_{o} ) = {\sum }_{i} {\sum }_{j} G ( t - τ ( r , θ , {x}_{o} , {x}_{ij} ) ) a ( r , θ , {x}_{o} , {x}_{ij} ) {s}_{ij} ( t - τ ( r , θ , {x}_{o} , {x}_{ij} ) )$$

The gain G(t) may have multiple programmable components including a static Low Noise Amplifier gain G_{LNA}, and a dynamic time-varying gain G_{TGC} (t) (also referred to as Time Gain Compensation) to compensate for tissue attenuation. The last gain stage may be an optional Programmable Gain Amplifier.

A low-pass filter (LPF) with a preferably programmable cut-off frequency provides antialiasing and improves signal to noise ratio (SNR). The multiple poles of the LPF can be distributed among the various gain stages.

The dynamic delay τ(r, θ, xₒ, xᵢⱼ) may vary with time to track the depth the echo is coming from as the transmit beam propagates deeper into the tissue. The input of the delay stage is a function of time, while the output of it is a function of depth (range). Depth is warped time due to the time varying delay.

The dynamic apodization or weight a(r, θ, xₒ, xᵢⱼ) may grow the active aperture size with depth to preserve resolution and tapers the contribution of edge elements, i.e., apodizes to reduce the beam sidelobes. For matrix arrays, the active aperture shape may also have an apodization effect. In some embodiments, the apodization weight is depth-dependent but it is binary, 0 for off and 1 for on, eliminating the need for a multiplication per element and per depth. By turning on elements around the beam origin within an ever-growing circle or ellipsoid a half-circle like apodization is achieved. The growth rate of the circle and ellipse may be controlled by a programmable f-number. Since G_{TGC} is applied before the delay operation, the gain may be dispersed in time as a function of the element-dependent delay. This can create an additional apodization effect for depths the gain changes rapidly.

The dynamic delay and weight computations may be performed by a computer given the beam parameters θ and xₒ, element coordinates xᵢⱼ, ADC sampling rate Fₛ, speed of sound c₀ and f-number. In many prior art systems, these computations are done fully or partially on remote processors.

The element sum stage may sum the time-aligned (therefore coherent) and weighted element signal.

Multiple beams with independent origins and angles can be generated in parallel using a duplicated set of delay, weight, and element sum stages. Alternatively, if the element data is stored for the full depth of interest, multiple beams can be formed serially using a single beamformer circuitry using the time in between transmit events, trading off frame rate.

### Array and Beam Geometry

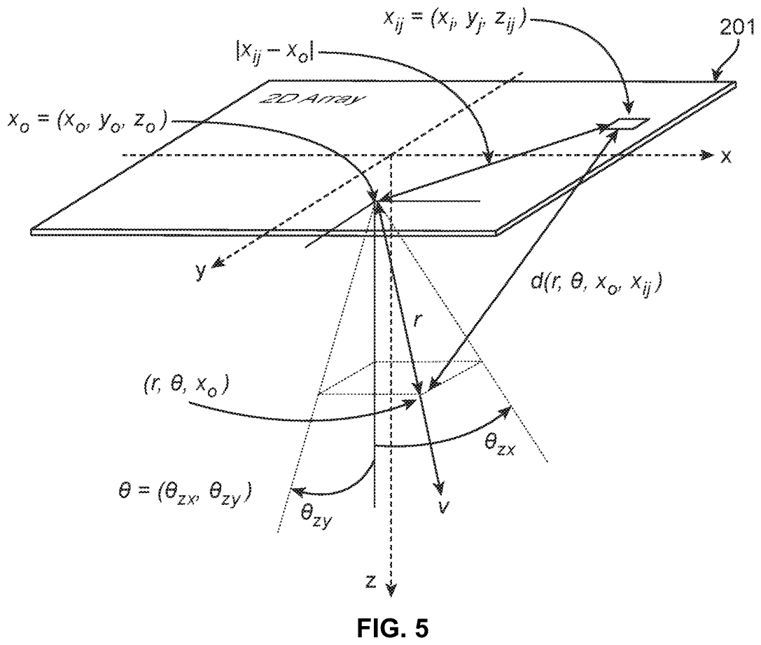

FIG. 5 shows a graph of the geometry of ultrasound beams generated by an ultrasound transducer array, according to some embodiments.

FIG. 5 depicts a Nₓ×N_{y}—element 2D array (201) on an x-y plane (or on a nonplanar curved x-y-z surface, which is not shown in FIG. 5) centered at the (0,0,0) of the Cartesian coordinates. xᵢⱼ is the x, y, and z coordinates (xᵢ, yⱼ, zᵢⱼ) of its (i, j)^{th} element. The elements of the 2D array can be on a square or rectangular grid, rotated square, rhombus (parallelogram), hexagonal, annular, or an arbitrary grid. The physical aperture can be a square, rectangle, circle or ellipse, or an arbitrary shape.

A beam can be defined in 3D by three parameters: a focusing depth r for the static transmit focus, or a set of focusing depths for the dynamic receive focus, a (nominal) beam origin xₒ which is a vector of its x, y, and z coordinates xₒ=(xₒ, yₒ, zₒ) and the angle θ which is also a vector of its z-x plane and z-y plane angles θ=(θ_{zx}, θ_{zy}). Note that bold letters are used here to represent vectors such as xₒ and θ. The coordinates of a sample along a receive beam (θ, xₒ) at depth (or range) r is (r, θ, xₒ). The convention for θ is such that θ_{zx} and θ_{zy} are positive from the +z axis to the +x and +y axes, respectively. The beam origin xₒ is also the depth zero (r=0). It is also the nominal center of the active aperture for beam (θ, xₒ) excluding the truncation by the physical aperture. All samples of the receive beam lie on the line the projections of which are at angles θ_{zx} and θ_{zy} on the z-x and z-y planes respectively.

2D imaging in the azimuth (i.e., x-z) plane is a special case where θ_{zy} and yₒ are zero for all beams. 2D imaging in the orthogonal elevation (y-z) plane corresponds to the case where θ_{zx} and xₒ are zero. A special case of 2D imaging is where the array is a 1-D array, e.g., N_{y}=1.

The geometry defined here can support independent combinations of scan geometries for the azimuth and elevation. For example, to define a Sector geometry both in azimuth and elevation, xₒ and yₒ would both be set to 0 for all beams. For a Linear scan, say in elevation, θ_{zy} would be set to zero for all beams while varying yₒ from the first row to the last. For a Vector format, such as in elevation, θ_{zy} would be varied from a negative angle to a positive one while varying yₒ from the first row to the last.

The geometry here can apply to multi-stage beamformation as well where a first stage subarray beamformer (micro beamformer) performs beamformation on groups of Sₓ× S_{y} elements, and a second stage Mₓ× M_{y} beamformer (macro beamformer) completes the beamformation on the outputs of the subarray beamformers where Nₓ=Sₓ Mₓ and N_{y}=S_{y} M_{y}.

In some examples, there are alternative coordinate systems to define the beams in 3D such as the Spherical Coordinates. The relationships between the angles (θ, φ) of the Spherical Coordinates centered at beam origin xₒ and the beam angles (θ_{zx}, θ_{zy}) of the framework adapted here are:

$${θ}_{zx} = {tan}^{- 1} ( tan θ sin φ ) \qquad [ 2 ]$$

$${θ}_{zy} = {tan}^{- 1} ( tan θ cos φ ) \qquad [ 3 ]$$

$$θ = {tan}^{- 1} ( {( {( tan {θ}_{zx} )}^{2} + {( tan {θ}_{zy} )}^{2} )}^{1 / 2} ) \qquad [ 4 ]$$

$$φ = {tan}^{- 1} ( \frac{tan {θ}_{zy}}{tan {θ}_{zx}} ) \qquad [ 5 ]$$

The analysis and derivations here can apply to any alternative beam definition with minor modifications.

### 3D Delay Equation

The distance d(r, θ, xₒ, xᵢⱼ) for depth r along the beam (θ, xₒ) for a particular element (i, j) can now be derived.

The Cartesian coordinates (bₓ, b_{y}, b_{z}) of the beam sample (r, θ, xₒ) are

$$( {b}_{x} , {b}_{y} , {b}_{z} ) = r ( {v}_{x} , {v}_{y} , {v}_{z} ) + ( {x}_{o} , {y}_{o} , {z}_{o} )$$

where, the unit vector v=(vₓ, v_{y}, v_{z}) along the beam is

$$v = ( {v}_{x} , {v}_{y} , {v}_{z} ) = \frac{( tan {θ}_{zx} , tan {θ}_{zy} , 1 )}{\sqrt{{tan}^{2} {θ}_{zx} , {tan}^{2} {θ}_{zy} , 1}}$$

and the x, y, z coordinates of the beam are

$${b}_{x} = {rv}_{x} + {x}_{o} ,$$

$${b}_{y} = {rv}_{y} + {y}_{o} ,$$

$${b}_{z} = {rv}_{z}$$

Then the distance between xᵢⱼ=(xᵢ, yⱼ) and (r, θ, xₒ)=(bₓ, b_{y}, b_{z}) is given by

$$d ( r , θ , {x}_{o} , {x}_{ij} ) = \sqrt{{( {x}_{i} - {b}_{x} )}^{2} + {( {y}_{j} - {b}_{y} )}^{2} + {( {z}_{ij} - {b}_{z} )}^{2}}$$

The square root of the summation of the squares of three terms can be written as the square root of the summation of the squares of two terms as follows.

$$d ( r , θ , {x}_{o} , {x}_{ij} ) = \sqrt{{( {x}_{i} - {b}_{x} )}^{2} + {( \sqrt{{( {y}_{j} - {b}_{y} )}^{2} + {( {z}_{ij} - {b}_{z} )}^{2}} )}^{2}}$$

Delay τ(r, θ, xₒ, xᵢⱼ) in s is the distance d(r, θ, xₒ, xᵢⱼ) in mm divided by the round-trip (two-way) speed of sound c₀ in mm/μs.

$$τ ( r , θ , {x}_{o} , {x}_{ij} ) = d ( r , θ , {x}_{o} , {x}_{ij} ) / ( {c}_{0} / 2 )$$

or in units of the number of samples at the ADC sampling rate of F_{S} in MHz

$$τ ( r , θ , {x}_{o} , {x}_{ij} ) = {F}_{s} d ( r , θ , {x}_{o} , {x}_{ij} ) / ( {c}_{0} / 2 )$$

### 3D Dynamic Delay and Weight Computer

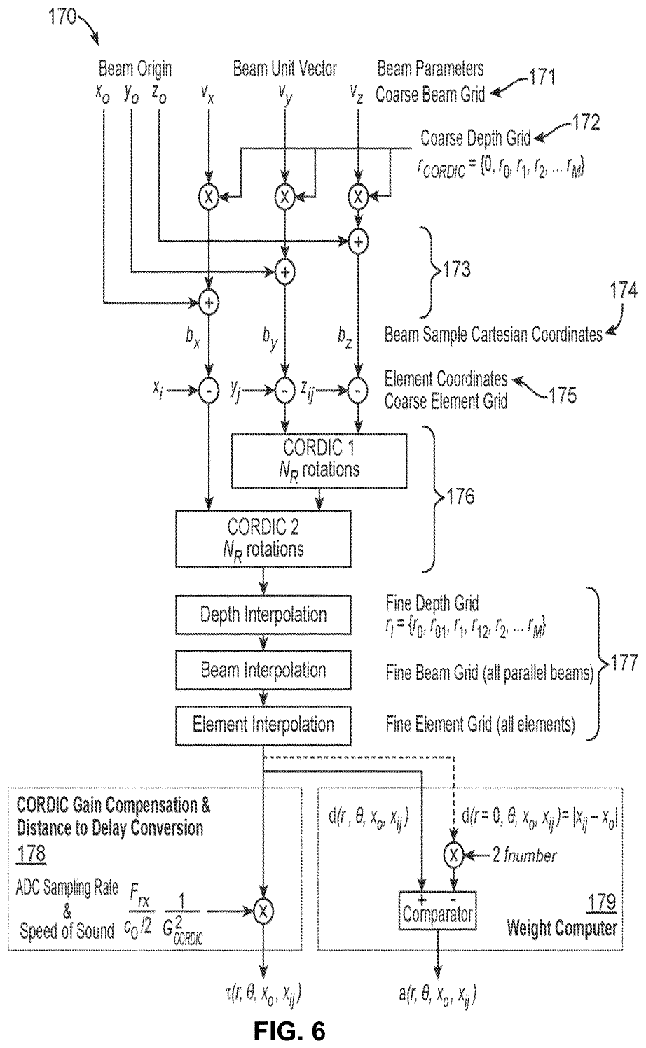

The delay formulation above lends itself to an efficient implementation using CORDIC (Coordinate Rotation Digital Computer) which is an efficient method to compute the square root of the squares of two numbers. FIG. 6 shows the block diagram and steps of a dynamic 3D delay and weight computer (170) using two cascaded CORDIC operations (176), according to some embodiments.

The inputs to the delay and weight computer may include the beams' origin, unit vector and focus depth(s), the coordinates of the elements, ADC sampling rate, the speed of sound, and f-number.

The beam unit vector Cartesian coordinates (171) may be multiplied with depth (172) and added to the beam origin coordinates (173) to create the beam sample Cartesian coordinates for a particular depth r (174). The x, y, and z coordinates of the elements may be subtracted from the respective x, y, and z coordinates of the beam sample (175) to create the inputs to the CORDIC operations. The output of the first CORDIC and the x component of the beam sample may form the inputs of the second CORDIC. The output of the second CORDIC may provide the distance between the element (i, j) and the beam sample (r, θ, xₒ) scaled by the gain of the two CORDIC stages (CORDIC is not a unit-gain operation). In a preferred implementation, the CORDIC gain compensation may be performed by the distance to delay conversion multiplier that is at the output of the delay computer (178).

In some embodiments, the cascaded CORDICs do 8 angle rotations each. This number of rotations may be sufficient to bring the maximum distance error within ±T₀/16, where T₀ is the period at the imaging center frequency F₀. Each angle rotation may take 2-bit shifts and 2 additions. For eight angle rotations, each CORDIC stage has a gain that is equal to around 1.65, and the two CORDIC stages together have a total gain of around 2.71.

In some examples, the CORDIC based high precision distance (delay) computations may be needed only for a sparse set of depths, elements, and beams. A linear interpolation between the CORDIC computed distance values (177) may be sufficient to keep the delay error within specifications. In some embodiments, the coarse range grid is spaced 4λ₀f_{number}², where λ₀ is the wavelength at imaging center frequency F₀. A linear distance interpolator may provide the distance values mid-point between the coarse range grid points. In some embodiments, CORDIC based delay computations are performed for a subset of beams, e.g., edge beams of a multibeam group, and a linear distance interpolator may provide the distance values for the in-between beams. In some embodiments, the coarse element grid is spaced 4 elements apart both in azimuth and elevation. Again, a linear distance interpolator may interpolate the distance values for the in-between elements. Since linear interpolations for powers of 2 up-sampling require only additions and bit shifts, they can be very efficient.

The last stage of the delay engine (178) may compensate for the non-unity gain of the CORDIC stages and converts the distance d(r, θ, xₒ, xᵢⱼ) which is in a unit of mm to delay τ(r, θ, xₒ, xᵢⱼ) in a unit of the ADC sample rate using the ADC sampling rate and speed of sound as the inputs. Having the distance to delay conversion at the very output may allow an easy way of optimizing the bulk speed of sound as a function of clinical application and the ADC sample rate as a function of the imaging center frequency. Alternatively, in some embodiments, the input parameters of CORDIC can be pre-compensated (pre-scaled) for the CORDIC gain and the distance to delay conversion factor by the Central Controller to eliminate the multiplications within the Delay and Weight Computer.

The orders of linear operations are interchangeable. For example, the distance to delay conversion can be done at any point in the delay computer signal path, or the interpolations can be reordered depending on implementation specific concerns.

In some embodiments, the weights are binary, i.e., an element is either on or off at any particular time/depth. The delay computer may provide the inputs to the weight computer. In some examples, the distance between any element and the beam origin can be computed by the delay computer by setting r to zero, |xᵢⱼ−xₒ|=d(r=0, θ, xₒ, xᵢⱼ). This distance scaled by a scalar that is a function of the f-number (aperture growth rate) can be compared to the distance output of the delay computer during receive event to turn each element on at the right time (depth) (179). With this method the aperture can be grown as a circle around the beam origin. Alternatively, both the growth rate and the aperture limits can be programmed for x and y independently, say for rectangular or ellipsoidal aperture growth.

### Data Acquisition

Data acquisition for all imaging modes and features can be generalized by a unifying concept of sampling in space, time, and parameter domains. This concept allows defining a Scan Design for any imaging mode or feature (B-Mode, Color Doppler, Spectral Doppler, M-Mode, Elastography, Pulse Inversion, Compounding, etc.) in any scan geometry (Sector, Trapezoid or Linear, cone, rectangular prism, etc.), in any number of dimensions (1D, 2D, 3D, 4D), as nested scan loops of events in space and time domains while varying a small set of parameters between events. An event can be a pulse-echo (transmit and receive), pulse only, or echo only event.

A planar or 2D image, for example, is formed by electronically scanning a transmit beam sequentially along the x or y axis, sampling the x-z or y-z plane. A volumetric or 3D image on the other hand is formed by a raster scan of a transmit beam along both the x and y axes, thus sampling the whole x-y-z space. Typically, the x axis, or azimuth, is the fast scan axis, while the y axis, or elevation, is the slow scan axis. In some use cases, the fast and slow axes can be rotated with respect to the x and y axes.

Receive beamformer forms multiple receive beams in parallel using echo received in response to each transmit event. The parallel receive beams may be distributed in x and/or y, typically centered around the transmit beam axis (line of sight).

The transmit and receive sampling grids in x and y can be spaced uniformly in beam angle, uniformly in sine of beam angle (denser closer to the z axis) or uniformly in beam origin. The spatial sampling alternatively can be on a nonlinear grid, e.g., hexagonal, spiral, etc.

A real-time 2D, or real-time 3D (e.g., 4D) image is formed by repeating a volumetric or planar scan described above at regular time intervals.

Modes for detection of motion or flow such as Color Doppler, Spectral Doppler and M-Mode require time domain sampling, where the object is sampled at regular time intervals at each line of sight (spatial location).

Modes to improve detail resolution, contrast resolution or penetration such as synthetic aperture, pulse inversion second harmonics, frequency compounding, spatial compounding, sequential transmit focus, etc., require parameter domain sampling, where the object is sampled multiple times as parameters such as aperture, phase, frequency, insonification angle or focus are varied. Parameter variations can be event-, frame- or volume-interleaved.

In a preferred embodiment, the concept of sampling in space, time, and parameter domains yields a mode- and feature-agnostic architecture and language for the Central Controller.

### Array and Beam Geometry

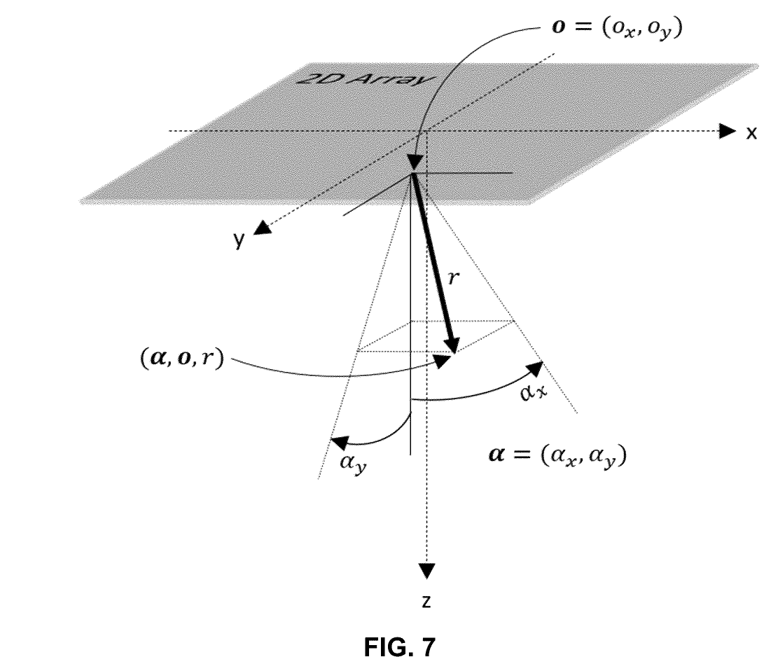

FIG. 7 shows a graph of the geometry of ultrasound beams generated by an ultrasound transducer array, according to some embodiments.

FIG. 7 shows a 2D array, with the Cartesian Coordinates centered at the center of the array where the x axis is the array's long axis (or azimuth), and the y axis is the array's short axis (or elevation). The object/tissue is assumed to be at z≥0. The beam axis whether it is a transmit or receive beam is defined by a pair of vectors, one for the angle and one for the origin, (α, o). The beam angle vector α can be defined by the angles between the z axis and the beam's projections on to the x-z and y-z planes, α=(ax, ay). The beam origin vector o of a beam can be defined by the x and y coordinates of the point where the beam crosses the 2D array surface, o=(oₓ, o_{y}). For planar transducer arrays the z coordinate of beam origins is zero.

The 3D space is sampled at regular depth intervals along a set (grid) of receive beams each with a unique angle and/or origin. The depth r of a spatial sampling point (α, o, r) on a receive beam (α, o) is defined as the distance between the beam origin and the spatial sampling point (α, o, r) (see the thick arrow).

### Scan Geometry

Imaging systems can be programmed to scan a single line, a plane (e.g., x-z plane), bi-plane (e.g., x-z and y-z planes), multiple planes, or a volume. Based on the beam grid used for spatial sampling, 2D scan geometry can be a sector, vector, trapezoid, linear, steered linear, etc., and 3D scan geometry can be pyramid, cone, truncated pyramid, truncated cone, rectangular prism, oblique rectangular prism, etc. For example, if all beams originate from the center of the array, the scan geometry is a sector, pyramid, or cone. If the beam origins are distributed across at least a part of the aperture while all beam angles are zero it is a linear scan geometry, or a rectangular prism. If the beam origins are distributed across the aperture while all beam angles are the same but nonzero, it is a steered linear or oblique rectangular prism. If the beam origins are distributed across the aperture while the beam angles monotonically vary in x and y, it is a trapezoidal scan geometry, truncated pyramid, or truncated cone. Hybrid scan geometries also exist, for example a 3D scan geometry can be a trapezoid in one of the lateral axes while linear along the orthogonal axis.

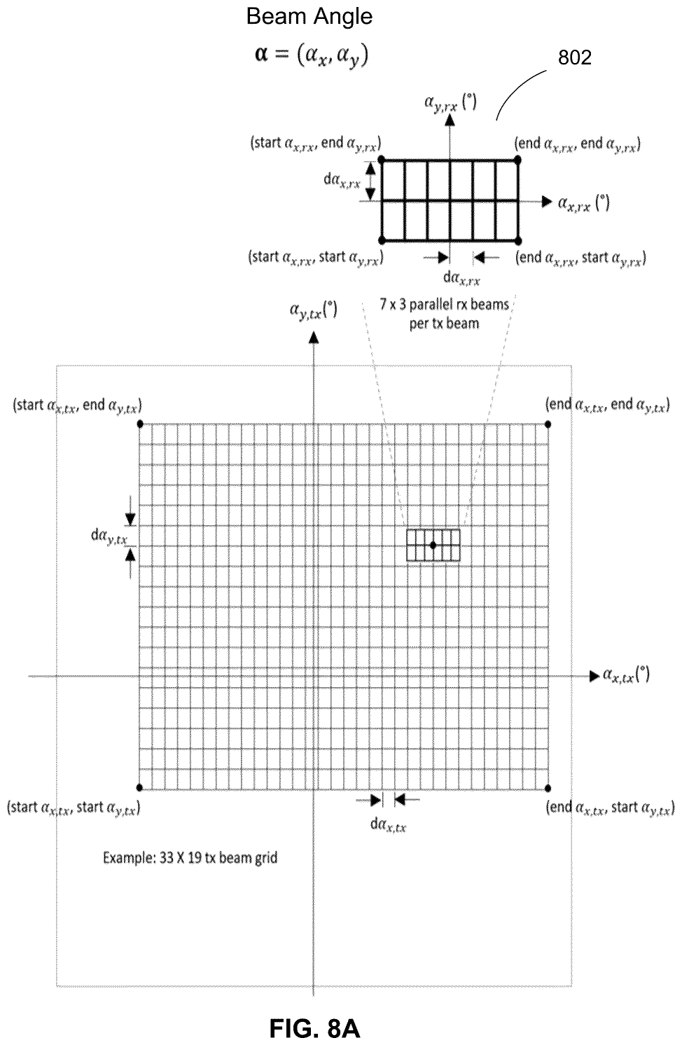

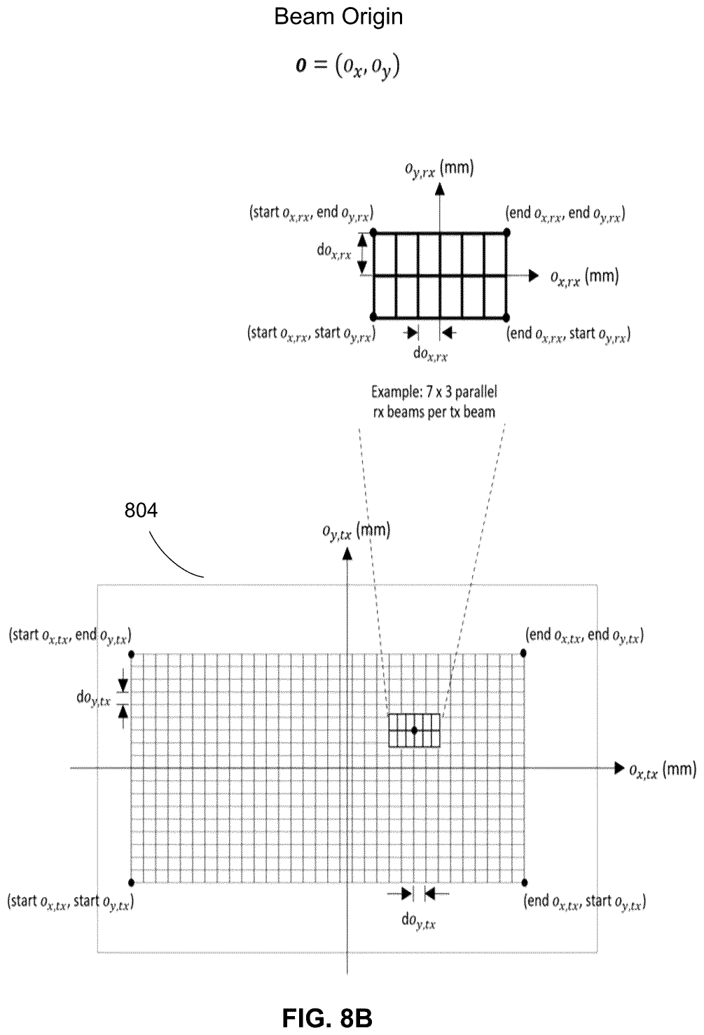

FIG. 8A and FIG. 8B illustrate an exemplary rectangular beam grid for sampling a truncated pyramid, according to some embodiments. Here a total of 627 transmit beams over a 33×19 beam grid (N_{x,tx}=33, N_{y,tx}=19) are uniformly distributed in angle and origin over independently programmable angular and origin domains that are off centered to indicate programmability of arbitrary volumes.

In this example, the transmit beam angles α_{x,tx} and α_{y,tx} start at one of the corners of the rectangular grid, (start α_{x,tx}, start α_{y,tx}) and increment uniformly with inter beam spacings dα_{x,tx} and dα_{y,tx}. These parameters along with the number of transmit beams in x and y, 33 and 19 in this example, define the angular span of the 3D FOV. The larger square box indicates the limits of steerability of the array (maximum angular domain) which is a function of the imaging center frequency, array's element spacing, and the effective element width.

FIG. 8A also shows a smaller rectangular grid 802 for the angles of a parallel receive beam group, the receive beams that are going to be formed in parallel by the receive beamformer in response to each transmit beam. Here a total of 21 parallel receive beams over a 7×3 beam grid (N_{x,rx}=7, N_{y,rx}=3) is centered at the angle of each transmit beam. For simplicity the parallel receive beam angle grid is shown only for one of the transmit beams. In this example, the parallel receive beam angles α_{x,rx} and α_{y,rx} start at (start α_{x,rx}, start α_{y,rx}) relative to (each) transmit beam angle and increment uniformly with inter beam spacings dα_{x,rx} and dα_{y,rx}.

FIG. 8B also shows an example for the beam origin grids of the same 33×19 transmit beams and the 7×3 parallel receive beams. The transmit and parallel receive beam origins are spaced uniformly in x and y, with inter-beam spacings do_{x,tx}, do_{y,tx}, do_{x,rx} and do_{y,rx}. The domain of the transmit beam origins is defined by one of the corners of the rectangular grid, (start o_{x,tx}, start o_{y,tx}), and the number of transmit beams in x and y, 33 and 19 in this example. The domain of the parallel receive beams relative to each transmit beam is in turn defined by (start o_{x,rx}, start o_{y,rx}), and the number of parallel receive beams in x and y, 7 and 3 in this example. The larger rectangle 804 shows the maximum domain of the beam origins. It is equal in size to the acoustic array.

In some examples, the receive beam angles/origins may or may not coincide with any of the transmit beam angles/origins. The angle/origin grids may also be distributed nonuniformly. The receive beam grid angles and origins may also be defined in absolute terms rather than relative to transmit beam angles/origins. All angle parameters here are in degrees and all origin parameters are in mm.

In some examples, by collapsing the 2D angle and/or origin grids to a 1D grid or to a single point, and or moving them around in the angular or origin domains one can define a wide range of scan geometries described earlier. For example, for a Sector, pyramidal or conical scan geometry all beam origins collapse to a single point at the center of the array (x, y)=(0, 0). For a Linear or rectangular prism scan geometry all beam angles collapse to a single point (ax, ay)=(0,0).

### Scan Design

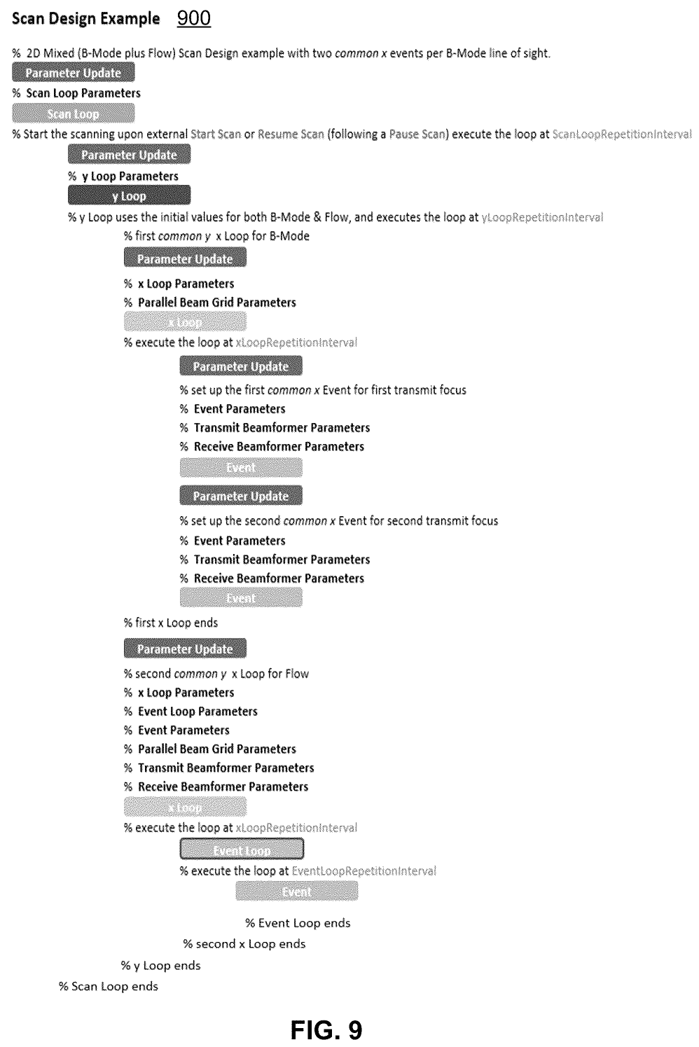

FIG. 9 illustrates an exemplary scan design, according to some embodiments. Referring to FIG. 9, a Scan design 900 is a serialized set of scan sequence instructions interspersed with timing and imaging parameters to be updated between instructions that can be read and interpreted by the Central Controller. The scan sequence instructions are Parameter Update, Scan Loop, y Loop, x Loop, Event Loop, and Event. Among these only the Parameter Update and Event are fundamental because all Scan Designs can be constructed by a sequence of these two instructions alone. But such a flattened Scan Design would take up too much memory and would be hard to read, understand and debug. The four looping instructions simplify and shorten Scan Designs while offering full flexibility.

In some embodiments, each Scan Design instruction can correspond to a given Central Controller state.

The Scan Design can start with a Parameter Update instruction to initialize some or all registers before the outermost loop, Scan Loop, starts. Scan Designs may use additional Parameter Update instructions before other instructions such as y Loop, x Loop, Event Loop, or Event. Reading this instruction, the Central Controller enters the Parameter Update state. In this state it fetches the parameters listed in between the Parameter Update instruction and the next instruction in the Scan Design and updates the values of the respective registers in the ASIC. A Scan Design may update a single parameter, multiple parameters, or all parameters at this state. Once the register updates are complete it reads the next instruction in the Scan Design.

The Scan Loop is the outermost loop of the scan sequence. For 2D and 3D imaging a Scan is a 2D frame or a 3D volume, respectively. A scan may comprise a single mode, or a mixed mode, e.g., B-Mode and Color Doppler. The Central Controller enters Scan Loop state upon reading the Scan Loop instruction and starts the scan or waits until it receives an external Start Scan signal before starting the scan. It repeats the Scan (frame, volume) indefinitely at a rate determined by Scan Loop Repetition Interval (the inverse of frame rate or volume rate), or a finite number of times determined by a Scan Count, or until an external End Scan signal. The Scan Loop Repetition Interval and Scan Count are input parameters that need to be updated before the Scan Loop instruction. The Start Scan and End Scan signals are initiated by an external processor, say upon a user request. The external processor may also pause and resume a scan loop using Pause Scan and Resume Scan signals.

The y Loop and x Loop are typically the slower (outer) loop and the faster (inner) loop controlling the raster scan of the lateral field of view. The parameters for these loops include the parameters of the transmit scan geometry and the loop repetition intervals. More specifically, the y Loop parameters are: N_{y,tz}, start α_{y,tx}, dα_{y,tz}, start o_{y,tx}, do_{y,tx}, and y Loop Repetition Interval and the x Loop parameters are: N_{x,tx}, start α_{x,tx}, dα_{x,tx}, start o_{x,tx}, do_{x,tx}, and x Loop Repetition Interval. In some examples, for a 2D image of an azimuthal plane (x-z plane), the y Loop parameters would set the angle and origin and repetition interval of a single y plane, while the x Loop sweeps the transmit beams from a starting angle and origin, with a given inter beam spacing N_{x,tx} times. The order of the x and y loops may be switched to make y loop the faster (inner) loop.

Before the inner loop the Parallel Beam Grid Parameters also need to be updated. They include N_{x,rx}, N_{y,rx}, start α_{x,rx}, start α_{y,rx}, dα_{x,rx}, dα_{y,rx}, start o_{x,rx}, start o_{y,rx}, do_{x,rx} and do_{y,rx}.

The Event Loop is reserved for temporal sampling of the object for motion/flow detection. It initiates a repeated set of events without any parameter updates in between events. Before any Event Loop the two Event Loop Parameters, Event Count and Event Loop Repetition Interval need to be updated. In some cases the Event Loop can be interleaved with the spatial loop (x Loop or y Loop) that it is inside of, for example for block-interleaved Color Doppler with long event repetition intervals.

The Central Controller enters its event execution state upon reading an Event instruction in the Scan Design. Before an Event the Event Parameters, Transmit Beamformer Parameters and Receive Beamformer Parameters need to be set using the Parameter Update instruction and parameter values. The Event Parameters may include Event Type (e.g., pulse-echo, pulse only, echo only), and Event Duration. The Transmit Beamformer Parameters may include transmit f-number, focus depth, and transmit pulse parameters. The Receive Beamformer Parameters may include receive analog front-end parameters, receive f-number, demodulation frequency, baseband filter parameters, etc.

In some Scan Designs, some states may be repeated multiple times. For example, there can be two Events within a x Loop, one for a shallow transmit focus and one for a deeper one. There can be two x Loops one after the other within a y Loop, one for B-Mode and one for Flow Mode for a frame interleaved mixed mode (see the example below). In some other Scan Designs, some of the states may not be used. For example, there may not be an Event Loop in a B-Mode only Scan Design.

A typical mixed mode Scan Design may take less than 1 k bits, even with complete reprogramming of the beamformer parameters before every common x and common y event.

A Scan Design example for a mixed mode is shown below. The Central Controller instructions (text with colorized backgrounds) define the scan sequence based on nested loops of events in space (x and y) and time, interspersed with parameters to be updated before each loop or event.

In this example there are two common y scans, one for B-Mode and one for Flow. For the first common y scan (B-Mode) there are two common x events, one for a shallow focus and one for a deep focus. In this example, following the initialization of all parameters, all x Loop parameters and Parallel Beam Grid Parameters are updated before each common y event, and all Tx and Rx Beamformer Parameters are updated before each common x event. Note however that in many situations only a subset of these parameters will require an update between common y and common x events. Hence the scan designs can be shorter than the example illustrated herein.

### Illustration of Subject Technology as Clauses

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. An imaging system integrated on a chip for autonomous scanning, comprising: an on-chip input memory configured to store scan sequence instructions and parameters; an on-chip processor configured to read the scan sequence instructions and parameters in the input memory; and an on-chip beamformer configured to be programmed and timed by the processor according to the scan sequence instructions and parameters.

Clause 2. The imaging system of Clause 1, wherein the imaging system is coupled to ultrasound transducers.

Clause 3. The imaging system of any of the preceding Clauses, wherein the chip is an Application Specific Integrated Circuit (ASIC).

Clause 4. The imaging system of any of the preceding Clauses, wherein the processor is configured to execute scanning autonomously, wherein the scanning includes a periodically repeated sequence of events.

Clause 5. The imaging system of Clause 4, wherein the sequence of events includes individual events, temporal loops of events, spatial loops of events in x and y, wherein each event and each loop of events from an inner most event loop, x loop, and y loop, to an outermost scan loop are timed.

Clause 6. The imaging system of any of Clauses 2-5, wherein the imaging system and the ultrasound transducers are packaged within a same ultrasound probe.

Clause 7. The imaging system of any of Clauses 2-6, wherein the imaging system and the ultrasound transducers are integrated.

Clause 8. The imaging system of any of the preceding Clauses, wherein the autonomous scanning includes beamforming processed by the beamformer.

Clause 9. The imaging system of any of the preceding Clauses, wherein the processor is an ultrasound-specific central controller.

Clause 10. The imaging system of Clause 9, wherein the ultrasound-specific central controller is configured to generate a scan sequence based on the scan sequence instructions and parameters.

Clause 11. The imaging system of Clause 9, wherein the ultrasound-specific central controller is configured to execute each event of transducers in a scan sequence based on the scan sequence instructions and parameters.

Clause 12. The imaging system of Clause 11, wherein the event is transmit and receive event, transmit only event, or receive only event.

Clause 13. The imaging system of any of the preceding Clauses, wherein the scan sequence instructions and parameters are provided by an external process outside of the chip.

Clause 14. The imaging system of any of the preceding Clauses, wherein the scan sequence instructions and parameters include timing for each event in a scan sequence.

Clause 15. The imaging system of any of the preceding Clauses, wherein the scan sequence instructions and parameters include imaging parameters for each event in a scan sequence.

Clause 16. The imaging system of any of the preceding Clauses, wherein the scan sequence instructions and parameters are included in multiple scan designs.

Clause 17. The imaging system of Clause 16, wherein each of the multiple scan designs are customized for a different imaging mode, feature, or clinical application that are selectable by a user.

Clause 18. The imaging system of any of the preceding Clauses, wherein the scan sequence instructions and parameters include programmable nested loops.

Clause 19. The imaging system of Clause 18, wherein each of the programmable nested loops corresponds to a transmit and/or receive event in a scan sequence.

Clause 20. The imaging system of Clause 19, wherein the transmit and/or receive event is preceded by imaging and timing parameters to be updated at that point in the scan sequence.

Clause 21. The imaging system of Clause 20, wherein the imaging and timing parameters to be updated are provided from an external user device.

Clause 22. The imaging system of Clause 20, wherein the imaging parameters are optimized according to the input memory.

Clause 23. The imaging system of any of Clauses 2-22, wherein the scan sequence instructions and parameters include space and time dimensions of transmit and/or receive events of the transducers.

Clause 24. The imaging system of any of the preceding Clauses, wherein the scan sequence instructions and parameters are for B-Mode.

Clause 25. The imaging system of any of the preceding Clauses, wherein the scan sequence instructions and parameters are for a mixed mode.

Clause 26. The imaging system of any of the preceding Clauses, wherein the scan sequence instructions and parameters include scan geometry.

Clause 27. The imaging system of Clause 26, wherein the scan geometry is for planar, bi-plane, or multi-plane imaging.

Clause 28. The imaging system of Clause 26, wherein the scan geometry is sector, vector, trapezoid, linear, or steered linear.

Clause 29. The imaging system of Clause 26, wherein the scan geometry is for 3D imaging or real-time 3D imaging.

Clause 30. The imaging system of Clause 26, wherein the scan geometry is a pyramid, cone, truncated pyramid, truncated cone, rectangular prism, or oblique rectangular prism.

Clause 31. The imaging system of any of the preceding Clauses, wherein the scan sequence instructions and parameters include instructions for sampling in space, time, and parameter domains.

Clause 32. The imaging system of any of the preceding Clauses, further comprising a detector coupled with the processor and the beamformer.

Clause 33. The imaging system of Clause 32, further comprising an output memory coupled with the detector and the processor.

Clause 34. The imaging system of Clause 33, further comprising a transceiver coupled to the output memory, the input memory, the processor and the beamformer.

Clause 35. The imaging system of Clause 34, wherein the transceiver coupled to an external computing device.

Clause 36. The imaging system of any of Clauses 2-35, wherein a number of the transducers are between 500 to 5000.

Clause 37. The imaging system of any of Clauses 2-36, wherein a number of the transducers are between 100 to 9000.

Clause 38. The imaging system of any of Clauses 2-37, wherein a number of the transducers are 2 k, wherein k is a non-negative integer.

Clause 39. The imaging system of any of the preceding Clauses, wherein the processor is configured to execute scan designs including the scan sequence instructions and parameters without any knowledge of a use case of scanning.

Clause 40. An imaging system integrated for autonomous scanning, comprising: an on-chip input memory configured to store scan design data; and an on-chip controller configured to read the scan design data in the input memory and to program and time beamforming of the transducers according to the scan design data.

Clause 41. An on-chip ultrasound imaging system, comprising: an on-chip controller configured to receive scan design data and to program and time beamforming according to the scan design data.

Clause 42. The imaging system of Clause 41, further comprising an on-chip input memory configured to store the scan design data.

Clause 43. The imaging system of any of Clauses 40-42, wherein the scan design data includes scan sequence instructions and parameters.

Clause 44. The imaging system of Clause 43, further comprising an on-chip beamformer configured to be programmed and timed by the controller to perform the beamforming according to the scan sequence instructions and parameters.

Clause 45. The imaging system of any of Clauses 40-44, wherein the imaging system is coupled to ultrasound transducers.

Clause 46. The imaging system of any of Clauses 40-45, wherein the on-chip ultrasound imaging system is Application Specific Integrated Circuit (ASIC).

Clause 47. The imaging system of Clause 45, wherein the imaging system and the ultrasound transducers are packaged within a same ultrasound probe.

Clause 48. The imaging system of Clause 45, wherein the imaging system and the ultrasound transducers are integrated.

Clause 49. The imaging system of any of Clauses 40-48, wherein the beamforming is included in an autonomous scanning process.

Clause 50. The imaging system of any of Clauses 40-49, wherein the controller is an ultrasound-specific central controller.

Clause 51. The imaging system of Clause 50, wherein the ultrasound-specific central controller is configured to generate a scan sequence based on the scan design data.

Clause 52. The imaging system of Clause 50, wherein the ultrasound-specific central controller is configured to execute each event of transducers in a scan sequence based on the scan design data.

Clause 53. The imaging system of Clause 52, wherein the event is transmit and receive event, transmit only event, or receive only event.

Clause 54. The imaging system of any of Clauses 40-53, wherein the scan design data are provided by an external process outside of the chip.

Clause 55. The imaging system of any of Clauses 40-54, wherein the scan design data includes timing for each event in a scan sequence.

Clause 56. The imaging system of any of Clauses 40-55, wherein the scan design data includes imaging parameters for each event in a scan sequence.

Clause 57. The imaging system of any of Clauses 40-56, wherein the scan design data include programmable nested loops.

Clause 58. The imaging system of Clause 57, wherein each of the programmable nested loops corresponds to a transmit and/or receive event in a scan sequence.

Clause 59. The imaging system of Clause 58, wherein the transmit and/or receive event is preceded by imaging and timing parameters to be updated at that point in the scan sequence.

Clause 60. The imaging system of Clause 59, wherein the imaging and timing parameters to be updated are provided from an external user device.

Clause 61. The imaging system of Clause 59, wherein the imaging parameters are optimized according to an input memory integrated within the on-chip ultrasound imaging system.

Clause 62. The imaging system of any of Clauses 40-60, wherein the scan design data includes space and time dimensions of transmit and/or receive events of the transducers.

Clause 63. The imaging system of any of Clauses 40-62, wherein the scan design data is for B-Mode.

Clause 64. The imaging system of any of Clauses 40-63, wherein the scan design data is for a mixed mode.

Clause 65. The imaging system of any of Clauses 40-64, wherein the scan design data includes scan geometry.

Clause 66. The imaging system of Clause 65, wherein the scan geometry is for planar, bi-plane, or multi-plane imaging.

Clause 67. The imaging system of Clause 65, wherein the scan geometry is sector, vector, trapezoid, linear, or steered linear.

Clause 68. The imaging system of Clause 65, wherein the scan geometry is for 3D imaging or real-time 3D imaging.

Clause 69. The imaging system of Clause 65, wherein the scan geometry is a pyramid, cone, truncated pyramid, truncated cone, rectangular prism, or oblique rectangular prism.

Clause 70. The imaging system of any of Clauses 40-69, wherein the scan design data includes instructions for sampling in space, time, and parameter domains.

Clause 71. The imaging system of any of Clauses 40-70, further comprising a detector coupled with the controller and the beamformer.

Clause 72. The imaging system of Clause 71, further comprising an output memory coupled with the detector and the controller.

Clause 73. The imaging system of Clause 72, further comprising a transceiver coupled to the output memory, an input memory, the controller and the beamformer, wherein the input memory is configured to store the scan design data.

Clause 74. The imaging system of Clause 73, wherein the transceiver coupled to an external computing device.

Clause 75. The imaging system of any of Clauses 40-74, wherein a number of the transducers are between 500 to 5000.

Clause 76. The imaging system of any of Clauses 40-75, wherein a number of the transducers are between 100 to 9000.

Clause 77. The imaging system of any of Clauses 40-76, wherein a number of the transducers are 2 k, wherein k is a non-negative integer.

Clause 78. The imaging system of any of Clauses 40-77, wherein the controller is configured to execute scan designs based on the scan design data without any knowledge of a use case of scanning.

Clause 79. A method for autonomous scanning, comprising: at an imaging system integrated on an Application Specific Integrated Circuit (ASIC) chip comprising an input memory and a processor: storing scan sequence instructions and parameters in the input memory; reading the scan sequence instructions and parameters in the input memory by the processor; and programing and timing beamforming by the processor according to the scan sequence instructions and parameters.

Clause 80. The method of Clause 79, wherein the imaging system is coupled to ultrasound transducers.

Clause 81. The method of Clause 80, wherein the imaging system and the ultrasound transducers are packaged within a same ultrasound probe.

Clause 82. The method of Clause 80, wherein the imaging system and the ultrasound transducers are integrated.

Clause 83. The method of any of Clauses 79-82, wherein the autonomous scanning includes beamforming processed by a beamformer.

Clause 84. The method of any of Clauses 79-83, wherein the processor is an ultrasound-specific central controller.

Clause 85. The method of Clause 84, further comprising generating a scan sequence based on the scan sequence instructions and parameters.

Clause 86. The method of Clause 84, further comprising executing each event of transducers in a scan sequence based on the scan sequence instructions and parameters.

Clause 87. The method of Clause 86, wherein the event is a transmit and receive event, transmit only event, or receive only event.

Clause 88. The method of any of Clauses 79-87, wherein the scan sequence instructions and parameters are provided by an external process outside of the chip.

Clause 89. The method of any of Clauses 79-88, wherein the scan sequence instructions and parameters include timing for each event in a scan sequence.

Clause 90. The method of any of Clauses 79-89, wherein the scan sequence instructions and parameters include imaging parameters for each event in a scan sequence.

Clause 91. The method of any of Clauses 79-90, wherein the scan sequence instructions and parameters include programmable nested loops.

Clause 92. The method of Clause 91, wherein each of the programmable nested loops corresponds to a transmit and/or receive event in a scan sequence.

Clause 93. The method of Clause 92, wherein the transmit and/or receive event is preceded by imaging and timing parameters to be updated at that point in the scan sequence.

Clause 94. The method of Clause 93, wherein the imaging and timing parameters to be updated are provided from an external user device.

Clause 95. The method of Clause 93, wherein the imaging parameters are optimized according to the input memory.

Clause 96. The method of any of Clauses 80-95, wherein the scan sequence instructions and parameters include space and time dimensions of transmit and/or receive events of the transducers.

Clause 97. The method of any of Clauses 79-96, wherein the scan sequence instructions and parameters are for B-Mode.

Clause 98. The method of any of Clauses 79-97, wherein the scan sequence instructions and parameters are for a mixed mode.

Clause 99. The method of any of Clauses 79-98, wherein the scan sequence instructions and parameters include scan geometry.

Clause 100. The method of Clause 99, wherein the scan geometry is for planar, bi-plane, or multi-plane imaging.

Clause 101. The method of Clause 99, wherein the scan geometry is sector, trapezoid, linear, or steered linear.

Clause 102. The method of Clause 99, wherein the scan geometry is for 3D imaging or real-time 3D imaging.

Clause 103. The method of Clause 99, wherein the scan geometry is a pyramid, cone, truncated pyramid, truncated cone, rectangular prism, or oblique rectangular prism.

Clause 104. The method of any of Clauses 79-103, wherein the scan sequence instructions and parameters include instructions for sampling in space, time, and parameter domains.

Clause 105. The method of Clause 104, wherein the imaging system further comprises a detector and a beamformer, wherein the detector is coupled with the processor and the beamformer.

Clause 106. The method of Clause 105, wherein the imaging system further comprises an output memory coupled with the detector and the processor.

Clause 107. The method of Clause 106, wherein the imaging system further comprises a transceiver coupled to the output memory, the input memory, the processor and the beamformer.

Clause 108. The method of Clause 107, wherein the transceiver coupled to an external computing device.

Clause 109. The method of any of Clauses 80-108, wherein a number of the transducers are between 100 to 9000.

Clause 110. The method of any of Clauses 80-109, wherein a number of the transducers are between 500 to 5000.

Clause 111. The method of any of Clauses 80-110, wherein a number of the transducers are 2 k, wherein k is a non-negative integer.

Clause 112. The method of any of Clauses 79-111, further comprising executing scan designs including the scan sequence instructions and parameters by the processor without any knowledge of a use case of scanning.

Clause 113. A non-transitory computer readable storage comprising any of the steps disclosed in any of the preceding Clauses.

Clause 114. A method comprising any of the steps disclosed in any of the preceding Clauses.

Clause 115. A system comprising one or more devices configured to perform any of the methods disclosed in any of the preceding Clauses.

Clause 116. The imaging system of any one of Clauses 1-78, wherein the chip comprises analog circuits for transmitting and receiving ultrasound signals.

Clause 117. The imaging system of any one of Clauses 1-40 and 42, wherein the on-chip input memory is factory programmed non-volatile memory.

Clause 118. The imaging system of any one of Clauses 1-39 and 43-44, wherein the scan sequence instructions and parameters are stored in a factory programmed nonvolatile input memory.

Clause 119. The method of any one of Clauses 79-112, wherein the chip comprises analog circuits for transmitting and receiving ultrasound signals.

Clause 120. The method of any one of Clauses 79-112, wherein the input memory of the chip is factory programmed non-volatile memory.

Clause 121. The method of any one of Clauses 79-112, wherein the scan sequence instructions and parameters are stored in a factory programmed nonvolatile input memory.

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

As used herein, the word “loop” or “component” refers to logic embodied in hardware or firmware, or to a collection of software instructions, possibly having entry and exit points, written in a programming language, such as, for example C++. A software loop or component may be compiled and linked into an executable program, installed in a dynamic link library, or may be written in an interpretive language such as BASIC. It will be appreciated that software loops or components may be callable from other loops or components or from themselves, and/or may be invoked in response to detected events or interrupts. Software instructions may be embedded in firmware, such as an EPROM or EEPROM. It will be further appreciated that hardware components may be comprised of connected logic units, such as gates and flip-flops, and/or may be comprised of programmable units, such as programmable gate arrays or processors. The loops or components described herein are preferably implemented as software loops or components, but may be represented in hardware or firmware.

It is contemplated that the loops or components may be integrated into a fewer number of loops or components. One loop or component may also be separated into multiple loops or components. The described loops or components may be implemented as hardware, software, firmware or any combination thereof. Additionally, the described loops or components may reside at different locations connected through a wired or wireless network, or the Internet.

In general, it will be appreciated that the processors can include, by way of example, computers, program logic, or other substrate configurations representing data and instructions, which operate as described herein. In other embodiments, the processors can include controller circuitry, processor circuitry, processors, general purpose single-chip or multi-chip microprocessors, digital signal processors, embedded microprocessors, microcontrollers and the like.

Furthermore, it will be appreciated that in one embodiment, the program logic may advantageously be implemented as one or more components. The components may advantageously be configured to execute on one or more processors. The components include, but are not limited to, software or hardware components, modules such as software modules, object-oriented software components, class components and task components, processes methods, functions, attributes, procedures, subroutines, segments of program code, drivers, firmware, microcode, circuitry, data, databases, data structures, tables, arrays, and variables.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

Although some of various drawings illustrate a number of logical stages in a particular order, stages that are not order dependent may be reordered and other stages may be combined or broken out. While some reordering or other groupings are specifically mentioned, others will be obvious to those of ordinary skill in the art, so the ordering and groupings presented herein are not an exhaustive list of alternatives. Moreover, it should be recognized that the stages could be implemented in hardware, firmware, software or any combination thereof.

It will also be understood that, although the terms first, second, etc. are, in some instances, used herein to describe various elements, these elements should not be limited by these terms. These terms are only used to distinguish one element from another. For example, a first transducer could be termed a second transducer, and, similarly, a second transducer could be termed a first transducer, without departing from the scope of the various described implementations. The first sensor and the second sensor are both sensors, but they are not the same type of sensor.

To the extent that the term “include,” “have,” or the like is used in the description or the claims, such term is intended to be inclusive in a manner similar to the term “comprise” as “comprise” is interpreted when employed as a transitional word in a claim.

As used herein, the term “comprising” indicates the presence of the specified integer(s), but allows for the possibility of other integers, unspecified. This term does not imply any particular proportion of the specified integers. Variations of the word “comprising,” such as “comprise” and “comprises,” have correspondingly similar meanings.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

What is claimed is:

1. An imaging system integrated on a chip for autonomous scanning, comprising:
an on-chip input memory configured to store scan sequence instructions and parameters;
an on-chip processor configured to read the scan sequence instructions and parameters in the input memory; and
an on-chip beamformer configured to be programmed and timed by the processor according to the scan sequence instructions and parameters.

2. The imaging system of claim 1, wherein the imaging system is coupled to ultrasound transducers, and wherein the imaging system and the ultrasound transducers are packaged within a same ultrasound probe.

3. The imaging system of claim 1, wherein the chip is Application Specific Integrated Circuit (ASIC).

4. The imaging system of claim 1, wherein the processor is configured to execute scanning autonomously, wherein the scanning includes a periodically repeated sequence of events.

5. The imaging system of claim 4, wherein the sequence of events includes individual events, temporal loops of events, spatial loops of events in x and y, wherein each event and each loop of events from an inner most event loop, x loop, and y loop, to an outermost scan loop are timed.

6. The imaging system of claim 1, wherein the autonomous scanning includes beamforming processed by the beamformer.

7. The imaging system of claim 1, wherein the processor is an ultrasound-specific central controller, wherein the ultrasound-specific central controller is configured to generate a scan sequence based on the scan sequence instructions and parameters, and/or to execute each event in a scan sequence based on the scan sequence instructions and parameters.

8. The imaging system of claim 1, wherein the scan sequence instructions and parameters include timing and/or imaging parameters for each event in a scan sequence.

9. The imaging system of claim 1, wherein the scan sequence instructions and parameters are included in multiple scan designs.

10. The imaging system of claim 9, wherein each of the multiple scan designs are customized for a different imaging mode, feature, or clinical application that are selectable by a user.

11. The imaging system of claim 1, wherein the scan sequence instructions and parameters include programmable nested loops.

12. The imaging system of claim 11, wherein each of the programmable nested loops corresponds to a transmit and/or receive event in a scan sequence, and/or wherein the transmit and/or receive event is preceded by imaging and timing parameters to be updated at that point in the scan sequence.

13. The imaging system of claim 2, wherein the scan sequence instructions and parameters include space and time dimensions of transmit and/or receive events of the transducers.

14. The imaging system of claim 1, wherein the scan sequence instructions and parameters include scan geometry, wherein the scan geometry is: (i) for planar, bi-plane, or multi-plane imaging, (ii) sector, vector, trapezoid, linear, or steered linear, (iii) for 3D imaging or real-time 3D imaging, or (iv) a pyramid, cone, truncated pyramid, truncated cone, rectangular prism, or oblique rectangular prism.

15. The imaging system of claim 1, wherein the scan sequence instructions and parameters include instructions for sampling in space, time, and parameter domains.

16. The imaging system of claim 1, wherein the processor is configured to execute scan designs including the scan sequence instructions and parameters without any knowledge of a use case of scanning.

17. An on-chip ultrasound imaging system, comprising an on-chip controller configured to receive scan design data and to program and time beamforming according to the scan design data.

18. The imaging system of claim 17, further comprising an on-chip input memory configured to store the scan design data.

19. A method for autonomous scanning, comprising:
at an imaging system integrated on an Application Specific Integrated Circuit (ASIC) chip comprising an input memory and a processor:
storing scan sequence instructions and parameters in the input memory;
reading the scan sequence instructions and parameters in the input memory by the processor; and
programing and timing beamforming by the processor according to the scan sequence instructions and parameters.

20. The method of claim 19, further comprising: (i) generating a scan sequence based on the scan sequence instructions and parameters, (ii) executing each event of transducers in a scan sequence based on the scan sequence instructions and parameters, (iii) executing scan designs including the scan sequence instructions and parameters by the processor without any knowledge of a use case of scanning, or any combination thereof.
